(19) United States
(12) Patent Application Publication
(10) Pub. No.: US 2007/0213535 A1
(43) Pub. Date: Sep. 13, 2007

(54) Process for the manufacture of aripiprazole by using purified carbostyril compounds such as 7-(4-halobutoxy)-3,4-dihydro-2(1H)-quinolinones

(75) Inventors: Michael Brand, RaAnana (IL); Irina Gribun, Bat-Yam (IL); Oded Arad, Rechovot (IL); Joseph Kaspi, Givatayim (IL)

Correspondence Address: Martin D. Moynihan;PRTSI, Inc., P.O. Box 16446, Arlington, VA, 22215, US

(73) Assignee: Chemagis Ltd., Bnei-Brak (IL)

(21) Appl. No.: 11/714,761

(22) Filed: Mar. 7, 2007

Related U.S. Application Data
(60) Provisional application No. 60/779,457, filed on Mar. 7, 2006.

(51) Int. Cl. C07D 403/02

(52) U.S. Cl. 544/363

(57) ABSTRACT

The present invention provides a process for purifying carbostyril derivatives such as 7-(4-bromobutoxy)-3,4-dihydro-2(1H)-quinolinone and 7-(4-chlorobutoxy)-3,4-dihydro-2(1H)-quinolinone and aripiprazole by passing a solution of the material in an organic solvent through a suitable absorbing material.

### RELATED APPLICATION

This application claims the benefit of U.S. Provisional Patent Application No. 60/779,457 filed on Mar. 7, 2006, the contents of which are incorporated herein by reference.

### FIELD OF THE INVENTION

The present invention relates to a process for the manufacture of aripiprazole by using purified carbostyril compounds such as 7-(4-halobutoxy)-3,4-dihydro-2(1H)-quinolinone

### BACKGROUND OF THE INVENTION

Aripiprazole (7-{4-[4-(2,3-dichlorophenyl)-1-piperazinyl]-butoxy}-3,4-dihydro-2(1H)-quinolinone) is represented by formula (I).

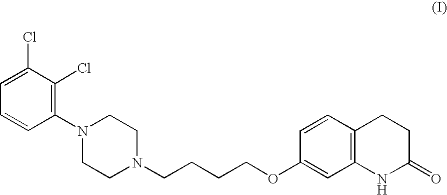

The drug is useful for treating schizophrenia and is available in tablets of different dosages, i.e., 5 mg, 10 mg, 15 mg, 20 mg and 30 mg and as a 1 mg/ml solution. Aripiprazole is marketed in the United States as Abilify™ by Bristol-Myers Squibb Company.

Several synthetic methods of aripiprazole preparation are described in U.S. Pat. No. 5,006,528 (hereinafter the '528 patent), including the method illustrated in Scheme 1.

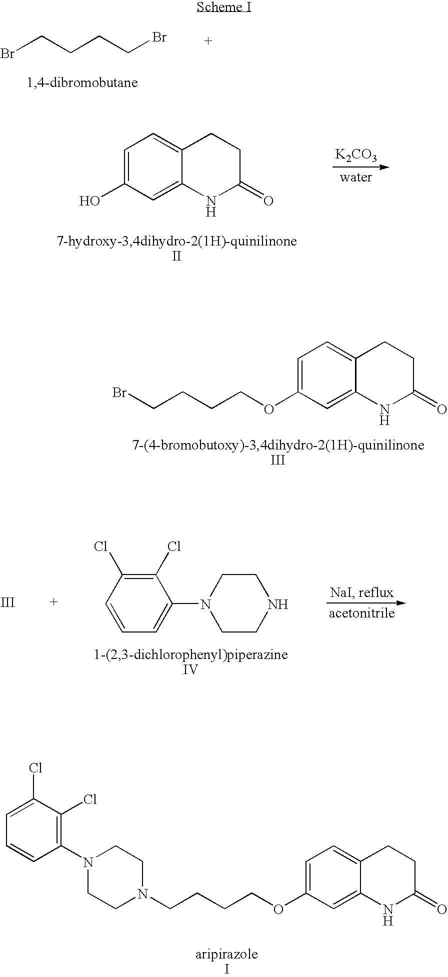

According to this synthetic method aripiprazole is prepared in two steps. The first comprises alkylating the hydroxy group of 7-hydroxy-3,4-dihydro-2(1H)-quinolinone (hereinafter 7-HQ) of formula (II) with 1,4-dibromobutane to obtain 7-(4-bromobutoxy)-3,4-dihydro-2(1H)-quinolinone of formula (III) (hereinafter 7-BBQ). A mixture of potassium carbonate, 7-HQ and 3 molar equivalents of 1,4-dibromobutane in water is refluxed for 3 hours. The reaction mixture thus obtained is extracted with dichloromethane, dried with anhydrous magnesium sulfate, and the solvent is removed by evaporation. The residue is purified by means of a silica gel column chromatography (eluent:dichloromethane), eluent evaporation and recrystallization from a mixture of ethanol and n-hexane to obtain 7-BBQ in 75.5% yield. The need to use a combination of methods (column chromatography and recrystallization) for purifying the carbostyril derivative 7-BBQ implies that it is difficult to obtain the compound in high purity.

In the second step 7-BBQ is reacted with 1-(2,3-dichlorophenyl)-piperazine of formula IV to obtain aripiprazole. Thus, a suspension of 7-BBQ and sodium iodide in acetonitrile is refluxed for 30 minutes. Triethylamine and 1-(2,3-dichlorophenyl)piperazine are added to the suspension and the reaction mixture is further refluxed for 3 hours. The solvent is then removed by evaporation, and the residue thus obtained is dissolved in chloroform, washed with water and dried over anhydrous magnesium sulfate. The solvent is removed by evaporation, and the residue is re-crystallized twice from ethanol to give aripiprazole having a melting point of 139.0-139.5° C.

A second method of preparing 7-BBQ is described by Oshiro Y. et al, J. Med. Chem. 1998, 41, 658-667, wherein 7-BBQ is obtained by reaction of 7-HQ with 3 molar equivalents of 1,4-dibromobutane in N,N-dimethylformamide (DMF) in the presence of potassium carbonate. The reaction is conducted by mixing the reagents for 4 hours at 60° C. followed by diluting with water. Ethyl acetate is added and the layers are separated and the organic phase is washed, dried, and evaporated to dryness in vacuum. Re-crystallization from ethanol gives 7-BBQ in 78% yield.

In U.S. Patent Application No. US 2006/0079689 (hereinafter the '689 application), which was filed on Oct. 11, 2005, titled “Processes for preparing and purifying carbostyril compounds such as aripiprazole and 7-(4-chlorobutoxy)-3,4-dihydro-2(1H)-quinolinone”, by the same applicant, which gains the benefit of U.S. Provisional Patent Application No. 60/617,073 filed on Oct. 12, 2004, and U.S. Provisional Patent Application No. 60/675,444 filed on Apr. 28, 2005, which are incorporated herein by reference in their entirety, processes are provided for preparing the carbostyril intermediate 7-(4-chlorobutoxy)-3,4-dihydro-2(1H)-quinolinone and aripiprazole thereof. According to the '689 application the purification of 7-(4-chlorobutoxy)-3,4-dihydro-2(1H)-quinolinone or 7-(4-bromobutoxy)-3,4-dihydro-2(1H)-quinolinone is carried out by slurrying process, which is liable in some cases to be less convenient for large scale preparation.

Therefore, there is still a need in the art for an improved low-cost process using purified carbostyril compounds such as 7-(4-chlorobutoxy)-3,4-dihydro-2(1H)-quinolinone or 7-(4-bromobutoxy)-3,4-dihydro-2(1H)-quinolinone (BBQ), which will be suitable for large-scale preparation, in terms of simplicity, chemical yield and purity of the product.

### SUMMARY OF THE INVENTION

According to an embodiment the present invention, there is provided a process for purifying carbostyril compounds comprising the steps of:
a) passing a solution containing the carbostyril compound in an organic solvent through a suitable absorbing material;
b) washing the absorbing material with a solvent;
c) combining the wash solvent with the solution containing the carbostyril compound; and
d) using the obtained solution in the next step to make aripiprazole.

The carbostyril compounds of the present invention can be 7-(4-halobutoxy)-3,4-dihydro-2(1H)-quinolinone, e.g., 7-(4-chlorobutoxy)-3,4-dihydro-2(1H)-quinolinone and 7-(4-bromobutoxy)-3,4-dihydro-2(1H)-quinolinone.

According to an aspect of the present invention, the suitable absorbing material is selected from the group consisting of aluminium oxide, Florisil®, Celite®, fumed silica gel, colloidal silica gel, chromatography grade silica gel, and combinations thereof. The presently most preferred suitable absorbing material is silica gel, which has an average particle size in the range of 40-200 microns, preferably silica gel 60, having particle size range of 40-63 microns.

In another embodiment, the process disclosed herein further comprises admixing the solution containing the purified carbostyril compound, that is the purified 7-(4-halobutoxy)-3,4-dihydro-2(1H)-quinolinone, (i.e., step (d)) with 1-(2,3-dichlorophenyl)piperazine mono hydrochloride and a base, e.g., potassium carbonate and optionally also a phase transfer catalyst e.g., tetra-butylammonium bromide to obtain aripiprazole.

The process disclosed herein may be applicable to purifying also aripiprazole, which is in itself also a carbostyril compound. Thus, in another embodiment, the process for purifying aripiprazole comprises the steps of:
a) passing a solution containing aripiprazole in an organic solvent through a suitable absorbing material;
b) washing the absorbing material with a solvent;
c) combining the wash solvent with the solution containing aripiprazole; and
d) isolating aripiprazole.

According to the present invention, isolating aripiprazole can be achieved e.g., by evaporating the solvent.

In another embodiment, the aripiprazole obtained by the process disclosed herein is having a purity of at least 98.5%, preferably having a purity over 99.5% (by HPLC).

### DETAILED DESCRIPTION OF THE INVENTION

The inventors of the present invention have repeated the synthetic procedure described in the '528 patent and found that relatively large amounts of impurities were obtained along with 7-BBQ. Among these impurities, the following were identified and isolated:
1. 1,4-bis[3,4-dihydro-2(1H)-quinolinone-7-oxy]butane (BQB) of formula (V);
2. N-(4-bromobutyl)-7-hydroxy-3,4-dihydro-2(1H)-quinolinone of formula (VI);
3. N-(4-bromobutyl)-7-(4-butoxy)-3,4-dihydro-2(1H)-quinolinone of formula (VII).
   1.

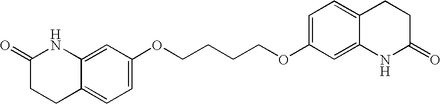

### 1,4-bis[3,4-dihydro-2(1H)-quinolinone-7-oxy]butane (BQB) (V)

2.

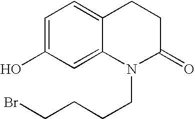

### N-(4-bromobutyl)-7-hydroxy-3,4-dihydro-2(1H)-quinolinone (VI)

3.

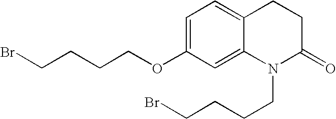

### N-(4-bromobutyl)-7-(4-butoxy)-3,4-dihydro-2(1H)-quinolinone (VII)

In a specific run it was found that 7-BBQ (III) prepared by the procedure described in the '528 patent, contained 10% of BQB, which could not be eliminated by re-crystallization, hence the only way to purify 7-BBQ was by column chromatography.

In view of the above mentioned results it is possible that the 75.5% yield of pure 7-BBQ obtained in the '528 patent is overstated.

The inventors of the present invention have further repeated the method of preparing 7-BBQ, described by Oshiro Y. et al, J. Med. Chem. 1998, 41, 658-667, and found that the reaction is very slow in these conditions (only about 40% of 7-BBQ is obtained after 19 hours) and that the reaction mixture contains substantial amounts of BQB.

Thus, in a search for an alternative process for purifying carbostyril compounds such as 7-(4-halobutoxy)-3,4-dihydro-2(1H)-quinolinone, the inventors of the present invention have designed and practiced a simple, low-cost and efficient process for purifying carbostyril compounds, which is provided herein. The process is suitable for large-scale preparation, in terms of simplicity, chemical yield and purity of the product.

The term purifying, as defined herein, can be any means of removing impurities from the 7-(4-halobutoxy)-3,4-dihydro-2(1H)-quinolinone compounds, including, but not limited to, crystallizing the 7-(4-halobutoxy)-3,4-dihydro-2(1H)-quinolinone, using chromatography or other separation techniques, extracting the 7-(4-halobutoxy)-3,4-dihydro-2(1H)-quinolinone from impurities, filtering the 7-(4-halobutoxy)-3,4-dihydro-2(1H)-quinolinone, or mixtures of any two or more of these techniques.

The 7-(4-halobutoxy)-3,4-dihydro-2(1H)-quinolinone can be, e.g., 7-(4-chlorobutoxy)-3,4-dihydro-2(1H)-quinolinone or 7-(4-bromobutoxy)-3,4-dihydro-2(1H)-quinolinone.

According to an embodiment the present invention, the process for purifying carbostyril compounds comprises the steps of:
a) passing a solution containing the carbostyril compound in an organic solvent through a suitable absorbing material;
b) washing the absorbing material with a solvent;
c) combining the wash solvent with the solution containing the carbostyril compound; and
d) optionally using the obtained solution in the next step to make aripiprazole.

According to an aspect of the present invention, the organic solvent is selected from the group consisting of toluene, ethyl benzene, xylenes, ethyl acetate, propyl acetate, isopropyl acetate, butyl acetate, isobutyl acetate, and mixtures thereof. The presently most preferred solvent is toluene.

According to another aspect of the present invention, the suitable absorbing material is selected from the group consisting of aluminium oxide, Florisil®, Celite®, fumed silica gel, colloidal silica gel, chromatography grade silica gel, and combinations thereof. The presently most preferred suitable absorbing material is silica gel, which has an average particle size in the range of 40-200 microns, preferably silica gel 60, having particle size range of 40-63 microns.

It is well known to skilled artisans in the field of preparative organic chemistry, that column chromatography is a laborious process, which consumes a lot of time (in collecting fractions, analyzing them and isolating the products), large volumes of solvents, and large quantities of the suitable absorbing material (e.g., silica gel) in a ratio of usually at least 1:30 (w/w) between the eluted organic mixture and the silica gel, which is needed to achieve good separation of the mixture's components. Thus, according to yet anther aspect of the present invention, the ratio between the carbostyril derivative and the silica gel, in the process provided herein, is less than 1:10 (w/w), preferable less than 1:5 (w/w), and more preferable less than 1:2 (W/w). In a preferred embodiment, the ratio between the carbostyril derivative and the silica gel is about 1:1 (w/w).

According to yet another aspect of the present invention, the purification is carried out at ambient temperature.

In another embodiment, the process disclosed herein further comprises admixing the solution containing the purified carbostyril compound, that is the purified 7-(4-halobutoxy)-3,4-dihydro-2(1H)-quinolinone, (i.e., step (d)) with 1-(2,3-dichlorophenyl)piperazine mono hydrochloride and a base, e.g., potassium carbonate and optionally also a phase transfer catalyst e.g., tetra-butylammonium bromide to obtain aripiprazole. The reaction of the carbostyril compound, with 1-(2,3-dichlorophenyl)piperazine mono hydrochloride and a base and optionally also a phase transfer catalyst to obtain aripiprazole is demonstrated in Scheme 2.

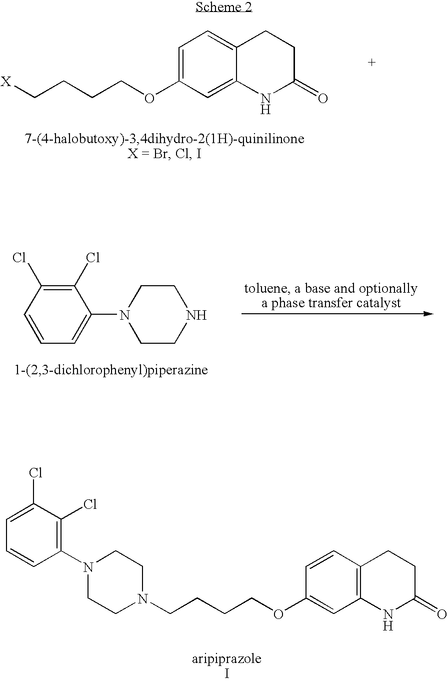

The process disclosed herein may be applicable to purifying also aripiprazole, which in itself is also a carbostyril compound. Thus, in another embodiment, the process for purifying aripiprazole comprises the steps of:
a) passing a solution containing aripiprazole in an organic solvent through a suitable absorbing material;
b) washing the absorbing material with a solvent;
c) combining the wash solvent with the solution containing aripiprazole; and
d) isolating aripiprazole.

According to an aspect of the present invention, the organic solvent is selected from the group consisting of toluene, ethyl benzene, xylenes, ethyl acetate, propyl acetate, isopropyl acetate, butyl acetate, isobutyl acetate, and mixtures thereof. The presently most preferred solvent is toluene.

According to the present invention, isolating aripiprazole can be achieved e.g., by evaporating the solvent.

In another embodiment, the aripiprazole obtained by the process disclosed herein is having a purity of at least 98.5%, preferably having a purity over 99.5% (by HPLC).

### EXAMPLES

### Example 1

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in 1-propanol in the presence of potassium carbonate

A mixture of 7-hydroxy-3,4-dihydro-2(1H)-quinolinone (40 g, 0.245 mole), 1,4-dichlorobutane (97% purity, 82.8 ml, 96.0 g, 0.735 mole. 3 equiv.) and potassium carbonate (37.24 g, 0.27 mole, 1.1 equiv.) in 1-propanol (400 ml) was heated under reflux for 10 hours (the reaction mixture contained 13.5% of BQB after reaction completion). The hot reaction mixture was then filtered and the solid was washed with hot 1-propanol (3×60 ml). The solvent and the excess of 1,4-dichlorobutane were removed by evaporation in vacuo. 2-Propanol (180 ml) was added to the thus obtained solid, and mixing was maintained at 5-10° C. for 3 hours. The solid was then collected by filtration, washed with cold 2-propanol (50 ml) and dried at 50° C. overnight to give crude 7-(4-chlorobutoxy)-3,4-dihydro-2(1H)-quinolinone (56.5 g, 91.0% yield, containing 11% of BQB).

### Example 2

### Purification of crude 7-(4-chlorobutoxy)-3,4-dihydro-2(1H)-quinolinone

A reaction vessel was charged with the crude 7-CBQ (18.0 g) of example 1 and toluene (270 ml). Stirring was applied for one hour at 25° C., and then the mixture was filtered. The toluene filtrate was passed with suction through a Buechner funnel containing a pad of silica gel 60, 40-63 microns, (18 g). Toluene (90 ml) was passed through the silica pad with suction. The two filtrates were combined and used in the next step of preparing crude Aripiprazole.

The BQB content was about 0.3% according to HPLC.

### Example 3

### Preparation of aripiprazole by reaction of 1-(2,3-dichlorophenyl)piperazine monohydrochloride with 7-(4-chlorobutoxy)-3,4-dihydro-2(1H)-quinolinone in the presence of phase transfer catalyst and potassium carbonate in a bi-phasic mixture containing toluene and water.

A reaction vessel was charged with the purified 7-(4-chlorobutoxy)-3,4-dihydro-2(1H)-quinolinone (CBQ) toluene solution of example 2 (about 350 ml), 1-(2,3-dichlorophenyl)piperazine mono hydrochloride (20.9 g, 0.078 mole), potassium carbonate (10.8 g, 0.078 mole), tetra-butylammonium bromide (2.1 g), and water (108 ml). The mixture was heated under reflux for 21 hours. Then, the reaction mixture was cooled to about 85° C. and toluene was added (270 ml) and stirring was maintained for 15 minutes. The phases were separated and water (91 ml) was added to the organic phase and the mixture was stirred at about 85° C. for 15 minutes after which time the layers were separated.

The apparatus was set up for azeotropic distillation in order to dry the toluene layer, and distillation was continued until the toluene distillate was clear. The majority of the toluene was distilled at atmospheric pressure to afford an oily residue, while the internal temperature had reached about 135° C. The mixture was cooled down to 85° C. and ethanol was added (270 ml) in portions at 85° C. to afford a solution. The solution was cooled to about 25° C. and stirred at that temperature for one hour. Then, the solution was cooled to about 5° C. and stirred at that temperature for one hour. The precipitate was collected by filtration and washed with ethanol to obtain a wet solid, which was dried at 60° C. to afford dry crude aripiprazole (21 grams, 65% yield), having a purity of 98%. The crude aripiprazole was crystallized twice from ethanol to obtain the crystallized material having a purity of 99.6% (containing less than 0.1% BQB)

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A process for preparing aripiprazole comprising admixing a solution containing a purified carbostyril compound, such as 7-(4-bromobutoxy)-3,4-dihydro-2(1H)-quinolinone or 7-(4-chlorobutoxy)-3,4-dihydro-2(1H)-quinolinone with 1-(2,3-dichlorophenyl)piperazine mono hydrochloride and a base, e.g., potassium carbonate and optionally also a phase transfer catalyst e.g., tetra-butylammonium bromide to obtain aripiprazole.

2. A process for purifying carbostyril compounds comprising the steps of:
a) passing a solution containing the carbostyril compound in an organic solvent through a suitable absorbing material;
b) washing the absorbing material with a solvent;
c) combining the wash solvent with the a solution containing the carbostyril compound; and
d) optionally using the obtained solution in the next step to make aripiprazole.

3. The process of claim 2, wherein the carbostyril compound is 7-(4-halobutoxy)-3,4-dihydro-2(1H)-quinolinone, that is 7-(4-bromobutoxy)-3,4-dihydro-2(1H)-quinolinone or 7-(4-chlorobutoxy)-3,4-dihydro-2(1H)-quinolinone.

4. The process of claim 2, wherein the organic solvent is selected from the group consisting of toluene, ethyl benzene, xylenes, ethyl acetate, propyl acetate, isopropyl acetate, butyl acetate, isobutyl acetate, and mixtures thereof.

5. The process of claim 4, wherein the organic solvent is toluene.

6. The process of claim 2, wherein the suitable absorbing material is selected from the group consisting of aluminium oxide, Florisil®, Celite®, fumed silica gel, colloidal silica gel, chromatography grade silica gel, and combinations thereof.

7. The process of claim 6, wherein the suitable absorbing material is silica gel 60, which has an average particle size in the range of 40-200 microns.

8. The process of claim 7, wherein the silica gel 60 has a particle size in the range of 40-63 microns.

9. The process of claim 8, wherein the ratio between the carbostyril derivative and the silica gel is less than 1:10 (w/w).

10. The process of claim 9, wherein the ratio between the carbostyril derivative and the silica gel is less than 1:5 (w/w).

11. The process of claim 10, wherein the ratio between the carbostyril derivative and the silica gel is about 1:1 (w/w).

12. The process of claim 2, wherein the purification is carried out at ambient temperature.

13. A process for purifying aripiprazole comprising the steps of:
a) passing a solution containing aripiprazole in an organic solvent through a suitable absorbing material;
b) washing the absorbing material with a solvent;
c) combining the wash solvent with the solution containing aripiprazole; and
d) isolating aripiprazole.

14. The process of claim 13, wherein the organic solvent is selected from the group consisting of toluene, ethyl benzene, xylenes, ethyl acetate, propyl acetate, isopropyl acetate, butyl acetate, isobutyl acetate, and mixtures thereof.

15. The process of claim 14, wherein the organic solvent is toluene.

16. The process of claim 13, wherein isolating aripiprazole can be achieved by evaporating the solvent.

17. The process of claim 1, wherein aripiprazole is obtained having a purity of at least 98.5% (by HPLC).

18. The process of claim 17, wherein aripiprazole is obtained having a purity equal to or higher than 99.5% (by HPLC).
